(19) United States
(12) Patent Application Publication
(10) Pub. No.: US 2026/0256457 A1
(43) Pub. Date: Sep. 3, 2026

(54) SYSTEM AND METHOD OF GENERATING AN ANATOMICAL THREE-DIMENSIONAL MODEL

(71) Applicant: Cyient Ltd., Hyderabad (IN)

(72) Inventor: Srinivas Rao Kudavelly, Bangalore (IN)

(73) Assignee: Cyient Ltd., Hyderabad (IN)

(21) Appl. No.: 19/540,972

(22) Filed: Feb. 16, 2026

(30) Foreign Application Priority Data
Mar. 3, 2025 (IN) 202541018615

(51) Int. Cl. A61B 8/00; G06T 7/00

(52) U.S. Cl. CPC A61B 8/5223; A61B 8/483; G06T 7/0012

(57) ABSTRACT

The present invention relates to a system 100 and a method 1100 for generating anatomical 3D models from ultrasound images. A first ML model of the system 100 determines anatomical features and corresponding dimensions from ultrasound images and generates a 3D model using shape priors based on nomogram data corresponding to gestational age. To enhance training and overcome data limitations of the first ML model, a second ML model generates synthetic ultrasound images using a generator-discriminator architecture. The system 100 automates anatomical assessments, ensuring accurate and consistent measurements. By integrating image segmentation, feature extraction, and synthetic data generation, the invention improves diagnostic precision and standardizes measurements.

### CROSS-REFERENCE TO RELATED APPLICATIONS

The present application claims priority under 35 U.S.C. § 119 to Indian Patent Application number 202541018615, filed Mar. 3, 2025, which is incorporated by reference herein in its entirety.

### FIELD OF INVENTION

The present invention generally relates to generation of 3D models. More specifically, the present invention is related to using machine learning for generating an anatomical 3D model of a human.

### BACKGROUND OF THE INVENTION

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

Anatomical analysis is a fundamental aspect of medical imaging, providing crucial information about the structure and development of various human organs, including fetal growth assessment during pregnancy. Accurate and consistent evaluation of anatomical parameters allows healthcare providers to monitor organ health, detect potential abnormalities, assess fetal development, and make informed clinical decisions in various medical fields. Traditional methods for anatomical analysis rely on manual assessments of key parameters by making use of ultrasound images. While these methods are well-established, they present following challenges and limitations.

One of the challenges is the time-intensive nature of manual analysis of the ultrasound images. The manual analysis involves identification of anatomical landmarks, and measurement of body parameters by a medical professional, such as a radiologist. Furthermore, ultrasound images are inherently complex, and patients or individuals (other than doctors) do not have the knowledge to interpret the ultrasound images. Unlike other imaging modalities, such as MRI or CT scans, ultrasound images are often grainy and lack clear contrasts, making it challenging for non-experts to recognize anatomical structures. This complexity can result in a lack of clarity and understanding for patients, leading to difficulties in comprehending their medical condition, foetal development, or potential abnormalities.

Hence, there is a growing need for a solution that can generate anatomical 3D models by making use of ultrasound images to assist medical professionals in examining ultrasound images more efficiently, reducing their workload while ensuring accuracy. Additionally, an intuitive and visually accessible representation of ultrasound images is required to bridge the gap between medical professionals and patients.

### SUMMARY OF THE INVENTION

This summary is provided to introduce aspects related to a method for generating an anatomical 3D model and the aspects are further described below in the detailed description. This summary is not intended to identify essential features of the claimed subject matter nor is it intended for use in determining or limiting the scope of the claimed subject matter.

In an embodiment, a method for generating an anatomical three-dimensional (3D) model is disclosed. The method comprises steps of receiving an actual gestational age and an ultrasound image of a human foetus. The method further comprises a step of determining one or more anatomical features and dimensions of the one or more anatomical features from the ultrasound image, by a first machine learning (ML) model. The first ML model is pre-trained using ultrasound images of foetuses. The method further comprises a step of selecting a shape prior from one or more shape priors related to different gestational ages of human foetuses, based on the actual gestational age of the human foetus. The one or more shape priors are pre-constructed using nomogram information. Furthermore, the method comprises a step of adapting the shape prior based on the dimensions of the one or more anatomical features to generate the anatomical 3D model of the human foetus.

In an aspect, the one or more anatomical features include biparietal diameter, head circumference, abdominal circumference, femur length, occipitofrontal diameter, cerebellar diameter, cisterna magna, nuchal translucency, humerus length, liver and spleen size, placental thickness and position, and tibia and fibula length.

In an aspect, the one or more anatomical features and the dimensions of the one or more anatomical features are determined by reducing, by an encoder of the first ML model, spatial resolution of the ultrasound image to determine one or more object features including edges, contours, boundaries, and shapes. Further, restoring, by a decoder of the first ML model, spatial resolution of the ultrasound image to provide segmented regions of interest. Furthermore, determining, by the decoder of the first ML model, dimensions of the one or more features in the segmented regions of interest. Furthermore, mapping the one or more object features and the dimensions of the one or more object features with a nomogram to determine the one or more anatomical features and the dimensions of the one or more anatomical features, wherein the nomogram includes information of biparietal diameter, head circumference, abdominal circumference, and femur length of foetuses.

In an aspect, the method further comprises steps of generating a report including at least one of, the ultrasound image comprising labels indicating the one or more anatomical features and the dimensions of the one or more anatomical features, and an evaluation of foetal growth levels by comparing the anatomical 3D model of the human foetus with nomogram information.

In an aspect, the first ML model is also pre-trained using synthetic ultrasound images generated by a second ML model.

In an aspect, the second ML model includes a pair of generator and discriminator. The generator utilizes an encoder-decoder architecture. The encoder-decoder architecture includes multi-scale skip connections to link layers of an encoder and a decoder to preserve fine-grained spatial details.

In an aspect, the synthetic ultrasound images are generated by the generator using foetal biometry obtained from a nomogram and at least one segmentation mask, wherein the foetal biometry comprises dimensions of anatomical features including biparietal diameter, head circumference, abdominal circumference, and femur length, and the at least one segmentation mask includes a spatial layout of the anatomical features.

In an aspect, generating the synthetic ultrasound images further comprises steps of providing, to the discriminator, the synthetic ultrasound images and real ultrasound images. Further, determining, by the discriminator, a loss occurred in generating the synthetic ultrasound images by comparing the synthetic ultrasound images with the real ultrasound images. Furthermore, iteratively tuning the generator until the loss falls below a pre-defined threshold value.

In an aspect, the first ML model and the second ML model are selected from a group consisting of a convolutional neural network, recurrent neural network, transformer, generative adversarial network, variational autoencoder, Simple Framework for Contrastive Learning of Visual Representations (SimCLR), and Bidirectional Encoder Representations from Transformers (BERT).

In an embodiment, a system for generating an anatomical 3D model is disclosed. The system comprises a processor and a memory. The memory is communicatively coupled with the processor. Further, the memory stores program instructions executable by the processor to receive an actual gestational age and an ultrasound image of a human foetus. Further, determine one or more anatomical features and dimensions of the one or more anatomical features from the ultrasound image, by a first machine learning (ML) model. The first ML model is pre-trained using ultrasound images of foetuses. Furthermore, select a shape prior from one or more shape priors related to different gestational ages of human foetuses, based on the actual gestational age of the human foetus. The one or more shape priors are pre-constructed using nomogram information. Furthermore, adapt the shape prior based on the dimensions of the one or more anatomical features to generate the anatomical 3D model of the human foetus.

In an aspect, the memory further stores program instructions configured to determine the one or more anatomical features and the dimensions of the one or more anatomical features by reducing, by an encoder of the first ML model, spatial resolution of the ultrasound image to determine one or more object features including edges, contours, boundaries, and shapes. Further, restoring, by a decoder of the first ML model, spatial resolution of the ultrasound image to provide segmented regions of interest. Furthermore, determining, by the decoder of the first ML model, dimensions of the one or more features in the segmented regions of interest. Furthermore, mapping, by the decoder of the first ML model, the one or more object features and the dimensions of the one or more object features with a nomogram to determine the one or more anatomical features and the dimensions of the one or more anatomical features, wherein the nomogram includes information of biparietal diameter, head circumference, abdominal circumference, and femur length of foetuses.

In an aspect, the memory further stores program instructions configured to generate, by the first ML model, a report including at least one of the ultrasound images comprising labels indicating the one or more anatomical features and the dimensions of the one or more anatomical features and an evaluation of foetal growth levels by comparing the anatomical 3D model of the human foetus with nomogram information.

In an aspect, the first ML model is also pre-trained using synthetic ultrasound images generated by a second ML model.

In an aspect, the second ML model includes a pair of generator and discriminator. The generator utilizes an encoder-decoder architecture. The encoder-decoder architecture includes multi-scale skip connections to link layers of an encoder and a decoder to preserve fine-grained spatial details.

In an aspect, the synthetic ultrasound images are generated by the generator using foetal biometry obtained from a nomogram and at least one segmentation mask, wherein the foetal biometry comprises dimensions of anatomical features including biparietal diameter, head circumference, abdominal circumference, and femur length, and the at least one segmentation mask includes a spatial layout of the anatomical features.

In an aspect, the memory further stores program instructions configured to provide, to the discriminator, the synthetic ultrasound images and real ultrasound images. Further, determine, by the discriminator, a loss occurred in generating the synthetic ultrasound images by comparing the synthetic ultrasound images with the real ultrasound images. Furthermore, iteratively tune the generator until the loss falls below a pre-defined threshold value.

In an embodiment, a method for generating an anatomical 3D model is disclosed. The method comprises steps of receiving an actual age and an ultrasound image of a human organ. The method further comprises a step of determining one or more anatomical features and dimensions of the one or more anatomical features from the ultrasound image, by a first machine learning (ML) model. The first ML model is pre-trained using ultrasound images of human organs. The method further comprises a step of selecting a shape prior from one or more shape priors related to different ages of human organs, based on the actual age of the human organ. The one or more shape priors are pre-constructed using nomogram information. Furthermore, the method comprises a step of adapting the shape prior based on the dimensions of the one or more anatomical features to generate the anatomical 3D model of the human organ.

In an aspect, the one or more anatomical features include ventricular ejection fraction, chamber dimensions, and wall thickness of human hearts, liver volume, portal vein diameter, and fat content of human livers, kidney size, cortical thickness, and renal pelvis dilation.

In an aspect, the first ML model is also pre-trained using synthetic ultrasound images generated by a second ML model.

In an aspect, the synthetic ultrasound images are generated by a generator of the second ML model using organ biometry obtained from a nomogram and at least one segmentation mask, wherein the organ biometry comprises dimensions of anatomical features including ventricular ejection fraction, chamber dimensions, and wall thickness of human hearts, liver volume, portal vein diameter, and fat content of human livers, kidney size, cortical thickness, and renal pelvis dilation, and the at least one segmentation mask includes a spatial layout of the anatomical features.

Other aspects and advantages of the invention will become apparent from the following description, taken in conjunction with the accompanying drawings, illustrating by way of example the principles of the invention.

### BRIEF DESCRIPTION OF THE DRAWINGS

The accompanying drawings constitute a part of the description and are used to provide a further understanding of the present disclosure. In the drawings:

FIG. 1 illustrates an architecture of a system for generating an anatomical three-dimensional (3D) model, in accordance with an embodiment of the present disclosure;

FIG. 2 illustrates a block diagram of the system for generating an anatomical 3D model, in accordance with an embodiment of the present disclosure;

FIG. 3 illustrates inputs, outputs, and functional elements of a first ML model used for generating a 3D model, in accordance with an embodiment of the present disclosure;

FIG. 4 illustrates a user interface of an annotation tool used for generating a 3D model, in accordance with an embodiment of the present disclosure;

FIG. 5 illustrates a detailed architecture of a classification layer of the first ML model, in accordance with an embodiment of the present disclosure;

FIG. 6 illustrates a detailed architecture of a segmentation layer of the first ML model, in accordance with an embodiment of the present disclosure;

FIG. 7A illustrates an exemplary output of the segmentation layer for an ultrasound image of a head, in accordance with an embodiment of the present disclosure;

FIG. 7B illustrates an exemplary output of the segmentation layer for an ultrasound image of an abdomen, in accordance with an embodiment of the present disclosure;

FIG. 7C illustrates an exemplary output of the segmentation layer for an ultrasound image of a femur, in accordance with an embodiment of the present disclosure;

FIG. 8 illustrates a block diagram showing working of a second ML model, in accordance with an embodiment of the present disclosure;

FIG. 9 illustrates a neural network architecture of the second ML model, in accordance with an embodiment of the present disclosure;

FIG. 10 illustrates a UI showing a 3D model and dimensions of a foetus, in accordance with an embodiment of the present disclosure; and

FIG. 11 illustrates a flow chart of a method of generating an anatomical 3D model, in accordance with an embodiment of the present disclosure.

A more complete understanding of the present invention and its embodiments thereof may be acquired by referring to the following description and the accompanying drawings.

### DETAILED DESCRIPTION OF THE INVENTION

Exemplary embodiments now will be described with reference to the accompanying drawings. The disclosure may, however, be embodied in many different forms and should not be construed as limited to the embodiments set forth herein; rather, these embodiments are provided so that this disclosure will be thorough and complete, and will fully convey its scope to those skilled in the art. The terminology used in the detailed description of the particular exemplary embodiments illustrated in the accompanying drawings is not intended to be limiting. In the drawings, like numbers refer to like elements.

It is to be noted, however, that the reference numerals used herein illustrate only typical embodiments of the present subject matter, and are therefore, not to be considered for limiting its scope, for the subject matter may admit to other equally effective embodiments.

The detailed description includes specific details for the purpose of providing a thorough understanding of the present invention. However, it will be apparent to those skilled in the art that the present invention may be practiced without these specific details.

The present invention introduces a system for generating anatomical 3D models from ultrasound images. It leverages machine learning (ML) to automate and enhance biometric measurements, ensuring accuracy and consistency. A first ML model processes ultrasound images to identify key anatomical features and determine their precise dimensions. To generate a 3D model, the system incorporates shape priors based on nomogram data, ensuring that the model aligns with expected growth patterns for a given gestational age.

To further enhance the reliability of the first ML model, the system includes a second ML model that generates synthetic ultrasound images. This model employs a generative approach to create high-quality training data, overcoming the limitations posed by insufficient real-world ultrasound datasets. The synthetic images help improve the robustness of the system, making it more adaptable to variations in ultrasound equipment, imaging conditions, and patient demographics.

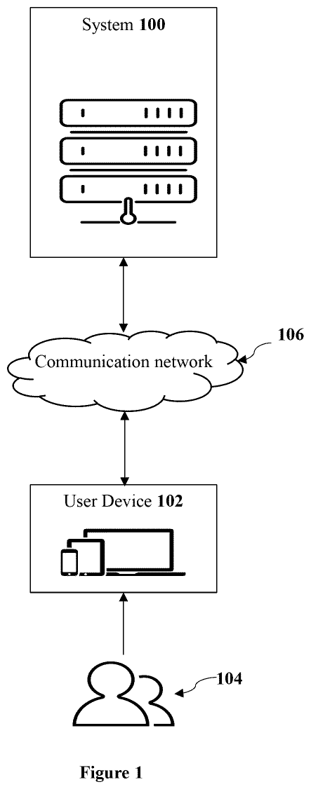

FIG. 1 illustrates a working architecture of a system 100 for generating an anatomical three-dimensional (3D) model, in accordance with an embodiment of the present disclosure. The system 100 may be a local or cloud-based server. A user may input one or more ultrasound images of a foetus to the system 100, using a user device 102. The user may further input an actual gestational age of the foetus to the system 100 to aid in anatomical 3D model generation.

Further, the system 100 may generate anatomical 3D models from the one or more ultrasound images provided by the user 104. The system 100 may be configured to analyze the one or more ultrasound images to define anatomical features and dimensions of the anatomical features present in the one or more ultrasound images. The system 100 may adapt a reference shape of a foetus, based on the anatomical features and dimensions of the anatomical features to generate the anatomical 3D model of the foetus.

The one or more ultrasound images may include, but are not limited to, 2D, 3D, 4D, Doppler, colour Doppler, power Doppler, elastography, transvaginal, transrectal, foetal echocardiography, intraoperative ultrasound, and contrast-enhanced ultrasound (CEUS). The user 104 may include but are not limited to healthcare professionals, researchers, and patients. Further, the user device 102 may include but are not limited to desktop computers, laptops, ultrasound machines, and mobile devices.

The system 100 may communicate with the user device 102, via a communication network 106. The communication network 106 may utilize network components to establish connection between the system 100 and the user device 102.

The network components may include hubs, switches, routers, bridges, and repeaters. The routers may be of different types such as Provide Edge (PE) routers, Customer Edge (CE) routers, and intermediate routers. The communication network 106 may be a wired and/or a wireless network. The communication network 106 may be implemented using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art.

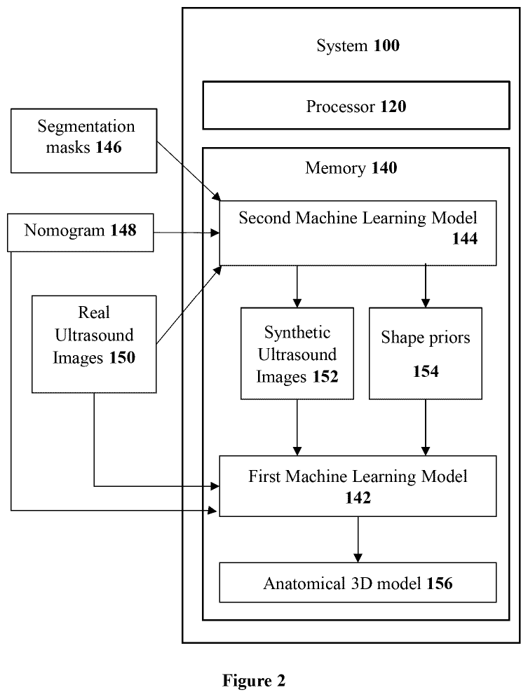

FIG. 2 illustrates a block diagram of the system 100 for generating an anatomical 3D model, in accordance with an embodiment of the present disclosure. The system 100 may include a processor 120 and a memory 140. The memory 140 may be communicatively coupled with the processor 120, and the memory 140 stores program instructions executable by the processor 120.

The processor 120 may include one or more general purpose processors (e.g., INTEL® or Advanced Micro Devices® (AMD) microprocessors) and/or one or more special purpose processors (e.g., digital signal processors or Xilinx® System On Chip (SOC) Field Programmable Gate Array (FPGA) processor), MIPS/ARM-class processor, a microprocessor, a digital signal processor, an application specific integrated circuit, a microcontroller, a state machine, or any type of programmable logic array.

The memory 140 may include, but is not limited to, non-transitory machine-readable storage devices such as hard drives, magnetic tape, floppy diskettes, optical disks, Compact Disc Read-Only Memories (CD-ROMs), and magneto-optical disks, semiconductor memories, such as ROMs, Random Access Memories (RAMs), Programmable Read-Only Memories (PROMs), Erasable PROMs (EPROMs), Electrically Erasable PROMs (EEPROMs), flash memory, magnetic or optical cards, or other type of media/machine-readable medium suitable for storing electronic instructions.

The memory 140 may include a plurality of storage locations that are addressable by the processor 120 for storing software programs and other necessary information (segmentation masks 146, nomogram 148, real and synthetic ultrasound images 150, 152, shape priors 154 and anatomical 3D models 156 generated by the system 100) associated with the embodiments described herein. The processor 120 may comprise hardware elements or hardware logic adapted to execute the software programs and manipulate data structures.

The memory 140 may include a first machine learning (ML) model 142. The first ML model 142 may be trained using training data including ultrasound images of foetuses. The ultrasound images used to train the first ML model 142 may be real ultrasound images 150 and synthetically generated ultrasound images 152 (hereinafter referred to as ‘synthetic ultrasound images 152’). Generation of synthetic ultrasound images 152 addresses a challenge of limited and non-standardized training data for the first ML model 142, hence increasing the accuracy and efficiency of the first ML model 142. The first ML model 142 may have an encoder-decoder based neural network architecture. In an embodiment, the first ML model 142 may be selected from a group consisting of a convolutional neural network, recurrent neural network, transformer, generative adversarial network, variational autoencoder, SimCLR, and BERT.

In an embodiment, the training data used to train the first ML model 142 may be collected from one or more clinical sites for Abdominal circumference (AC), Head circumference (HC) and Femur length (FL) segmentation. The training data may involve data of several operators using different ultrasound machines that may add another layer of diversity to the training data.

A user 104 may provide one or more ultrasound images of a foetus and an actual gestational age of the foetus to the first ML model 142, via a user device 102. The first ML model 142 may determine one or more anatomical features and corresponding dimensions from the one or more ultrasound images.

The anatomical features may include, but are not limited to, biparietal diameter, head circumference, abdominal circumference, femur length, occipitofrontal diameter, cerebellar diameter, cisterna magna, nuchal translucency, humerus length, liver and spleen size, placental thickness and position, and tibia and fibula length. The anatomical features are essential for anatomical labelling in the one or more ultrasound images of the foetus.

Further, the first ML model 142 may select a shape prior 154 based on the actual gestational age of the foetus, provided by the user 104. The first ML model 142 may then generate the anatomical 3D model 156 from the one or more ultrasound images, by adapting the shape prior 154 based on the anatomical features and dimensions of the anatomical features. The anatomical 3D model 156 may be provided to the user 104 via the user device 102. The 3D model 156 may enhance the ability of the user 104 to assess foetal development more efficiently.

Shape priors may be reference 3D models that may represent a usual shape and structure of a foetus at different stages of gestation, based on accepted medical data such as nomograms. The shape priors may help in creation of an accurate 3D model by providing a reference for an expected size and proportions of the foetus at a given age. Further, nomogram may be graphical tools or charts that may represent a relationship between various anatomical features and the gestational age of a foetus. Nomogram may include information on how certain measurements, such as biparietal diameter or femur length, are expected to change as the foetus develops.

In the system 100, nomogram 148 may be used to provide a reference for the expected growth patterns of the foetus, helping to guide selection of shape priors based on the gestational age of the foetus. The first ML model 142 may use the nomogram 148 to compare the anatomical 3D model 156 with expected growth benchmarks, ensuring that biometric measurements align with standard foetal development patterns.

In an embodiment, the first ML model 142 may produce a report for clinical evaluation. The report may include annotated ultrasound images with labels marking the anatomical features and their corresponding dimensions. Additionally, the report may provide an evaluation of foetal growth levels. The evaluation may provide a comparison between the anatomical 3D model 156 and nomogram 148 information to determine whether growth of the foetus is normal. The report may be displayed to the user 104 via the user device 102. Further, the user 104 may be able to edit the information in the report. Such reports enhance the ability of medical professionals to assess foetal development more efficiently.

The system 100 may include a second ML model 144 to generate the synthetic ultrasound images 152 used to train the first ML model 142. Generation of synthetic ultrasound images 152 addresses the challenge of limited and non-standardized training data for the first ML model 142, hence increasing the accuracy and efficiency of the first ML model 142. Furthermore, the second ML model 144 may be selected from a group consisting of a convolutional neural network, recurrent neural network, transformer, generative adversarial network, variational autoencoder, SimCLR, and BERT. The second ML model 144 may be trained using data including segmentation masks 146, nomogram 148, and real ultrasound images 150 of foetuses.

A segmentation mask may be a representation that may highlight specific areas or features within an image including boundaries or regions of interest. The segmentation masks 146 may be used to identify and separate different anatomical features of the foetus in the ultrasound image. The segmentation masks 146 may be created based on known shapes of anatomical features and may help the system 100 focus on relevant parts of the image, including head, abdomen, or limbs, to determine corresponding dimensions accurately. By using the segmentation masks 146, the system 100 may improve the precision of the biometric measurements and ensure that the 3D model is built from the right parts of the image. Further, nomogram 148 may include information on how certain measurements, like biparietal diameter or femur length, are expected to change as the foetus develops. In an embodiment, the second ML model 144 may generate segmentation masks 146 based on nomogram 148 and real ultrasound images 150.

In an embodiment, the second ML model 144 may be further configured to generate shape priors 154. The shape priors 154 may be stored in the memory 140 to be used by the first ML model 142 to generate the anatomical 3D model 156. Rendering algorithms may help interpolate spatial details to create a 3D representation of a human foetus, at different gestational ages. Furthermore, the rendering algorithms may provide volume shading, depth perception, and texture mapping, to generate shape priors 154.

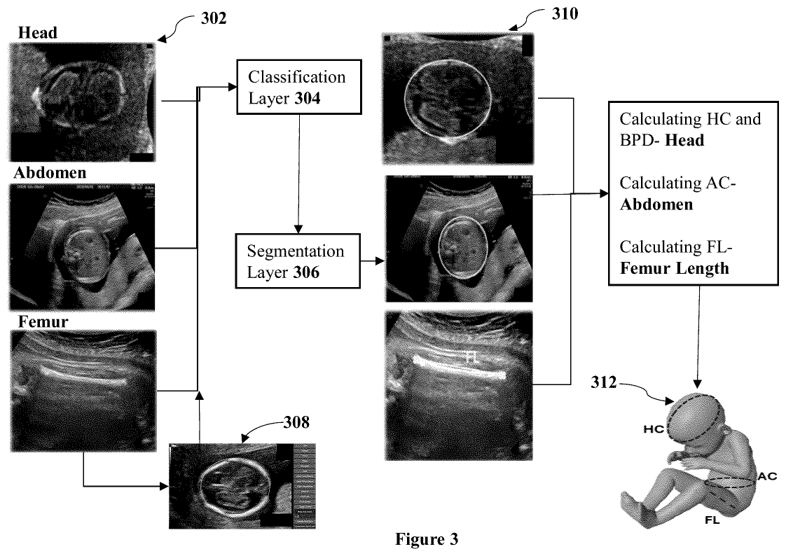

FIG. 3 illustrates inputs, outputs, and functional elements of the first ML model 142, in accordance with an embodiment of the present disclosure. The first ML model 142 receives one or more ultrasound images 302 including but not limited to ultrasound images of head, abdomen and femur of a foetus. Further, the first ML model 142 may determine one or more anatomical features and corresponding dimensions from the one or more ultrasound images 302 by performing image classification and segmentation. The classification and segmentation may enable precise image annotation for identifying the anatomical features and extracting dimensions of the anatomical features with high accuracy and consistency.

The classification process of the first ML model 142 may involve categorizing the one or more ultrasound images into different segmented regions of interest. Classification may ensure that subsequent processing steps are applied within the correct anatomical context, allowing for more accurate feature extraction.

Once the classification is complete, the first ML model 142 may analyze the segmented regions of interest and identify the anatomical features of the foetus. The first ML model 142 may perform segmentation by assigning labels to specific anatomical features within the segmented region of interest to provide segmented images 310. Segmentation may involve defining boundaries of different anatomical features within the ultrasound image. For example, distinguishing the skull, brain, and facial bones within a head scan, or identifying the liver, stomach, and abdominal wall in an abdominal scan. The segmentation step may enable the system 100 to extract precise anatomical measurements such as biparietal diameter, head circumference, abdominal circumference, femur length, and other anatomical features that may serve as foetal growth indicators.

The segmentation labels in the segmented images 310 serve as a foundation for image annotation, where the ultrasound image may be enhanced with visual markers indicating the location and dimensions of the anatomical features. Annotations may help medical professionals quickly interpret the ultrasound results, reducing manual effort and minimizing potential errors caused by operator variability. Additionally, the segmented and annotated images contribute to the generation of an anatomical 3D model, where the anatomical features may be used to adapt a shape prior of the foetus. By leveraging both classification and segmentation, the first ML model 142 automates ultrasound image analysis, ensuring accurate, consistent, and efficient anatomical measurement.

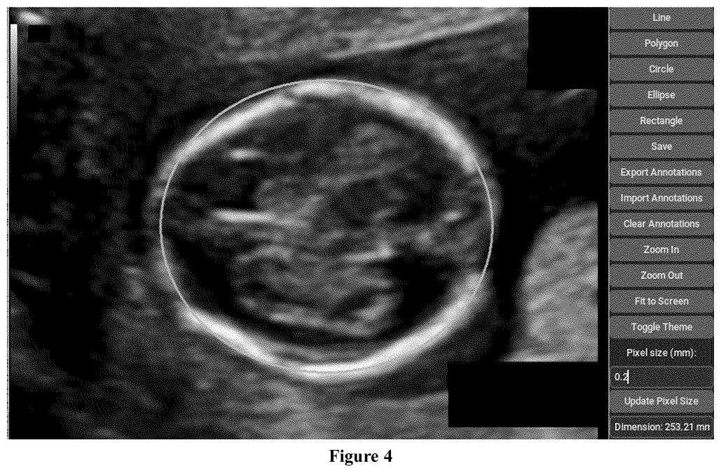

In an embodiment, the first ML model 142 may include an annotation tool 308 for image annotation, as illustrated in FIG. 4. The annotation tool 308 may be a software application presented to the user via the user device 102 to label specific regions or features within the ultrasound image. Labels, or annotations, may take various forms such as lines, polygons, circles, ellipses, and rectangles may be used to highlight, measure, and categorize different elements. The annotations may be saved in a structured format including, but not limited to, JSON. The ultrasound images with the annotations may then be provided to the first ML model 142 for classification and segmentation.

The classification and segmentation are implemented using a classification layer 304 and a segmentation layer 306 of the first ML model 142 respectively. In an embodiment, the system 100 may allow the user to modify the classifications and labels in the segmented images, via the user device 102.

Once the one or more ultrasound images are converted into segmented images 310 including segmented regions of interest and labels for anatomical features and dimensions of anatomical features, the first ML model 142 generates the 3D anatomical model 312. The 3D anatomical model 312 may be generated by selecting a shape prior 154 from one or more shape priors 154 stored in the memory 140, based on the actual gestational age provided by the user 104. The first ML model 142 may then adapt the shape prior 154 based on the dimensions of the one or more anatomical features to generate the anatomical 3D model 312 of the foetus. The anatomical 3D model 312 may include annotations of the anatomical features and corresponding dimensions.

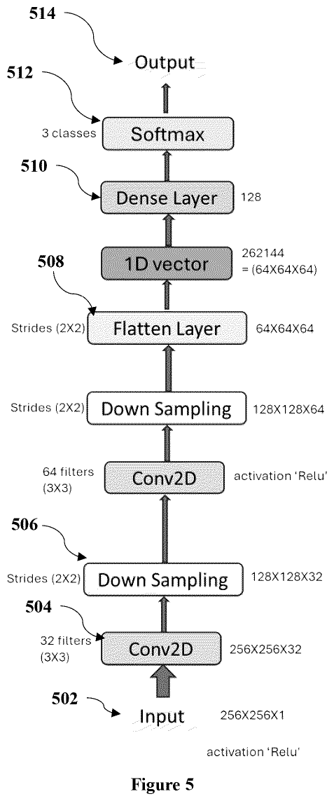

FIG. 5 illustrates a detailed architecture of the classification layer 304 of the first ML model 142, in accordance with an embodiment of the present disclosure. The classification layer 304 of the first ML model 142 may follow a convolutional neural network (CNN) architecture. The classification layer 304 may classify the one or more ultrasound images 302 into segmented regions of interest such as the head, abdomen, and femur. The classification layer 304 may begin with an input layer 502, which may receive the one or more ultrasound images 302.

The next stage may be a plurality of convolutional layers 504 (Conv2D) that may extract object features such as edges, contours, and textures. The convolutional layers 504 may apply filters to scan the one or more ultrasound images 302 and detect anatomical features. Following each convolutional layer, a down-sampling layer 506 (implemented using max-pooling function) may be employed to reduce spatial dimensions while retaining most relevant object features.

Once a plurality of rounds of convolution 504 and down-sampling 506 are performed with various numbers of filters, a map of the object features may be passed to a flatten layer 508. The flatten layer 508 may convert multi-dimensional data into a one-dimensional vector. Such conversion enables further processing in a fully connected (dense) layer 510, where the first ML model 142 learns high-level representations of the ultrasound image and interprets the object features. Finally, a softmax activation function 512 in the output layer 514 assigns probabilities to different classes, allowing the model to classify the image into appropriate segmented regions of interest anatomically.

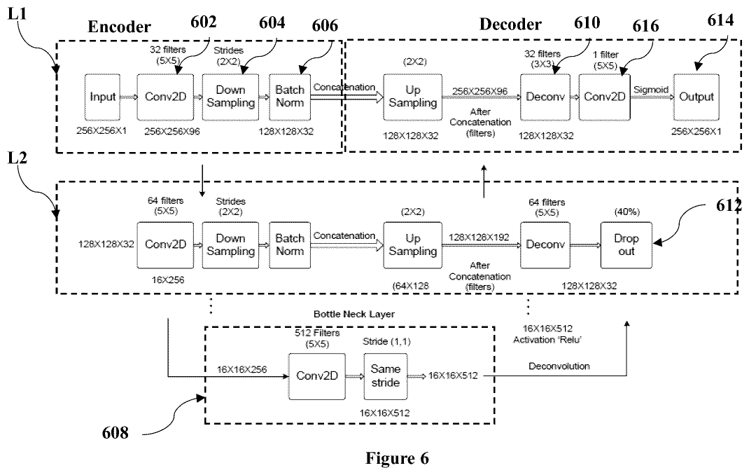

FIG. 6 illustrates a detailed architecture of the segmentation layer 306 of the first ML model 142, in accordance with an embodiment of the present disclosure. The segmentation layer 306 of the first ML model 142 may perform image segmentation to determine the anatomical features in the one or more ultrasound images 302 using an encoder-decoder based convolutional neural network (CNN) architecture.

The encoder may follow a downsampling approach where the spatial dimensions of the one or more ultrasound images 302 are progressively reduced while extracting the object features. This may be achieved through convolutional layer 602 (Conv2D) with large kernel sizes, which may detect edges, textures, and spatial patterns. Each convolutional layer is followed by a LeakyReLU (Leaky Rectified Linear Unit) activation function in a down-sampling layer 604 to introduce non-linearity while preventing vanishing gradients. Convolutional layers typically perform linear transformations, therefore, to allow the first ML model 142 to learn complex patterns, we need a non-linear activation function such as the LeakyReLU activation function.

Further, batch normalization layer 606 may be applied to stabilize training and accelerate convergence. Batch normalization may enhance neural network training by addressing internal covariate shift, where distribution of activations may change as training progresses, leading to instability in training. By normalizing activations within each mini-batch through mean and variance adjustments, batch normalization may ensure that activations remain within a stable range, potentially preventing drastic weight updates and mitigating issues such as exploding or vanishing gradients. Additionally, batch normalization may accelerate convergence by maintaining well-scaled and centered activations, allowing the model to learn more efficiently. This may enable the use of higher learning rates, facilitating faster optimization and potentially reducing the number of training epochs required to achieve optimal performance.

Furthermore, instead of traditional max-pooling, strided convolutions may employed for downsampling. This may enable the first ML model 142 to learn object features while preserving crucial anatomical details. The number of filters of convolutional layer and deconvolutional layer may increase at each level (from L1, L2 (as shown in the FIG. 6) to . . . Ln), allowing the first ML model 142 to capture both fine and abstract features essential for accurate segmentation.

A bottleneck layer 608 may serve as a transition point between the encoder and decoder, operating at lowest resolution while preserving most abstract and meaningful object features. A Conv2D layer extracts object features, followed by a ReLU activation to enhance the ability of the first ML model 142 to determine anatomical features in ultrasound images. The Conv2D layer ensures that the most significant spatial features are retained before reconstruction process begins.

The decoder may follow an upsampling path that may gradually restore the spatial resolution of the one or more ultrasound images, reconstructing the segmented regions of interest and including the anatomical features with high precision. This may be achieved through deconvolutional layers 610 (Conv2DTranspose), which may increase spatial dimensions and restore fine-grained details. Dropout layers 612 may be incorporated to prevent overfitting, ensuring that the first ML model 142 may generalize well across diverse ultrasound images. Batch normalization may be used at each stage to normalize activations, further improving training stability and efficiency. A key feature of the first ML model 142 is the use of skip connections, which may directly link corresponding layers of the encoder and decoder. The skip connections ensure that fine spatial details extracted during encoding process are preserved in the upsampling stage, leading to more precise segmentation.

The output layer 614 may consist of a Conv2DTranspose layer 616 with a sigmoid activation function, which generates a segmented image 310 including a pixel-wise segmentation mask in the segmented regions of interest. The segmentation mask defines the anatomical features within the one or more ultrasound images 302, ensuring that the corresponding dimensions derived from the anatomical features are accurate and clinically reliable. The combination of strided convolutions, skip connections, dropout layers, and batch normalization ensures robustness against variations in ultrasound imaging conditions, making the system 100 adaptable to different ultrasound equipment and patient demographics.

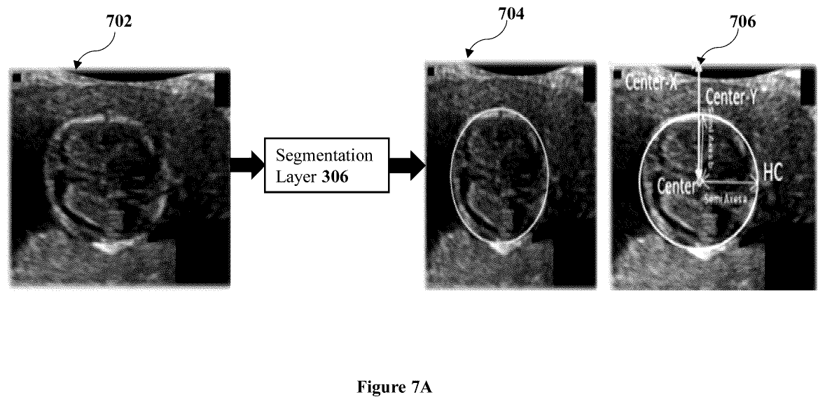

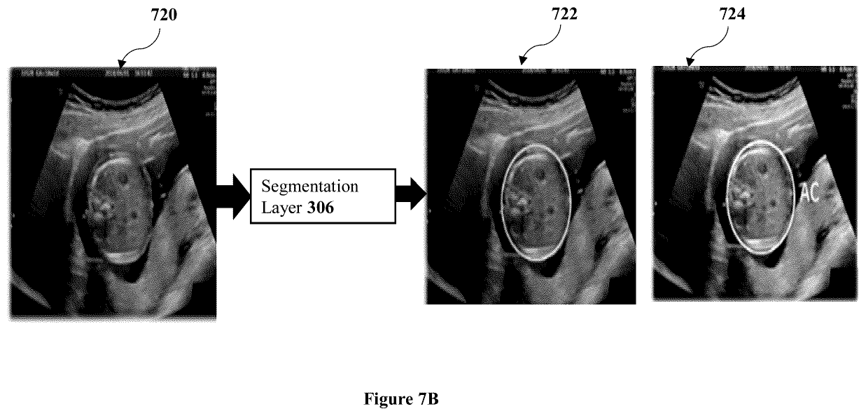

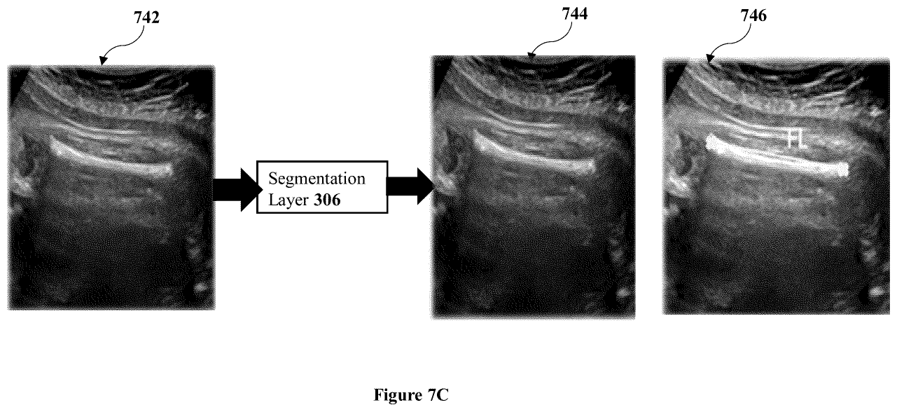

FIG. 7A illustrates an exemplary output of the segmentation layer 306 for an ultrasound image 702 of a head, in accordance with an embodiment of the present disclosure. The output illustrates a segmented region of interest 704 and a segmented image 706 including dimensions such as head circumference (HC) and biparietal diameter (BPD).

FIG. 7B illustrates an exemplary output of the segmentation layer 306 for an ultrasound image 722 of an abdomen, in accordance with an embodiment of the present disclosure. The output illustrates a segmented region of interest 724 and a segmented image 726 including dimensions such as abdominal circumference (AC).

FIG. 7C illustrates an exemplary output of the segmentation layer 306 for an ultrasound image 742 of a femur, in accordance with an embodiment of the present disclosure. The output illustrates a segmented region of interest 744 and a segmented image 746 including dimensions such as femur length (FL).

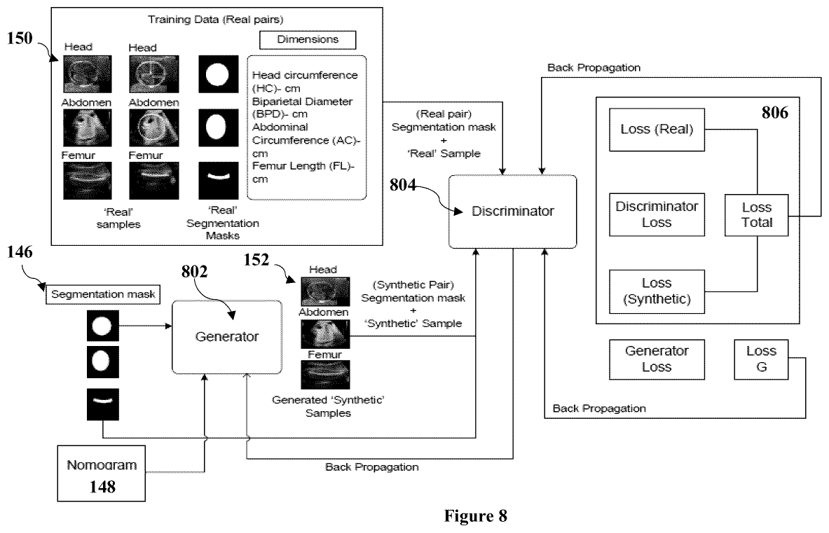

FIG. 8 illustrates working of the second ML model 144, in accordance with an embodiment of the present disclosure. The synthetic ultrasound images 152 used to train the first ML model 142, may be generated by the second ML model 144. To generate the synthetic ultrasound images 152, the second ML model 144 uses a generator-discriminator architecture. A generator 802 may use an encoder-decoder structure with multi-scale skip connections to ensure anatomical consistency with input parameters. The discriminator may evaluate the realism and fidelity of generated images with respect to conditioned inputs.

The generator 802 of the second ML model 144 may receive nomogram 148 of foetal biometry. The nomogram 148 may include, but is not limited to, anatomical proportions of head circumference, abdominal circumference and femur length. Further, the generator may receive segmentation masks 146 which may represent spatial layouts of the foetal biometry. A segmentation mask may be a representation that may highlight specific areas or features within an image including boundaries or regions of interest. The segmentation masks 146 may be used to identify and separate different anatomical features of the foetus in the ultrasound image. The generator 802 may then produce synthetic ultrasound images by combining the nomogram 148 and the segmentation masks 146.

The discriminator 804 in the generator-discriminator architecture may leverage a PatchGAN-based approach to combine both global and local discrimination capabilities. The discriminator 804 may receive real ultrasound images, the synthetic ultrasound images generated by the generator 802, the nomogram 148 and the segmentations masks. By employing patch-level discrimination, the discriminator 804 evaluates small regions of the synthetic ultrasound images, ensuring that local textures and fine details resemble those found in real ultrasound images. At the same time, global evaluation may assess overall structure and coherence of the synthetic ultrasound images, ensuring that the synthesized ultrasound adheres to anatomical consistency.

The discriminator 804 may utilize a loss function 806. The loss function 806 may output a single scalar value that may represent a likelihood of the synthetic ultrasound image being interpreted as real or synthetic. The output may include discriminator loss, generator loss (Loss G), and total loss (Loss Total). The discriminator loss consists of two components, real loss (Loss (Real)) and synthetic loss (Loss (Synthetic)). The real loss measures ability of the discriminator ability to correctly classify real data, while the synthetic loss quantifies its ability to correctly identify generated data as fake. The generator loss measures effectiveness of the generator in producing realistic data that can fool the discriminator. The total loss consists of both the real and synthetic discriminator losses, with variations depending on specific architectures used. Additional refinements, such as gradient penalties or auxiliary losses, may be introduced to improve training stability and enhance the quality of synthetic data generation.

Further, the second ML model 144 may be trained using a combination of adversarial and reconstruction losses. Adversarial loss may help the generator to produce images that are indistinguishable from real ultrasound images. Reconstruction loss may minimize pixel-wise differences between the synthetic ultrasound images and real ultrasound images. Additionally, a perceptual loss may help the generator to create images with realistic textures by comparing high-level features extracted from a pre-trained network. This multi-loss strategy enables the second ML model 144 to generate synthetic ultrasound images that are visually and anatomically realistic.

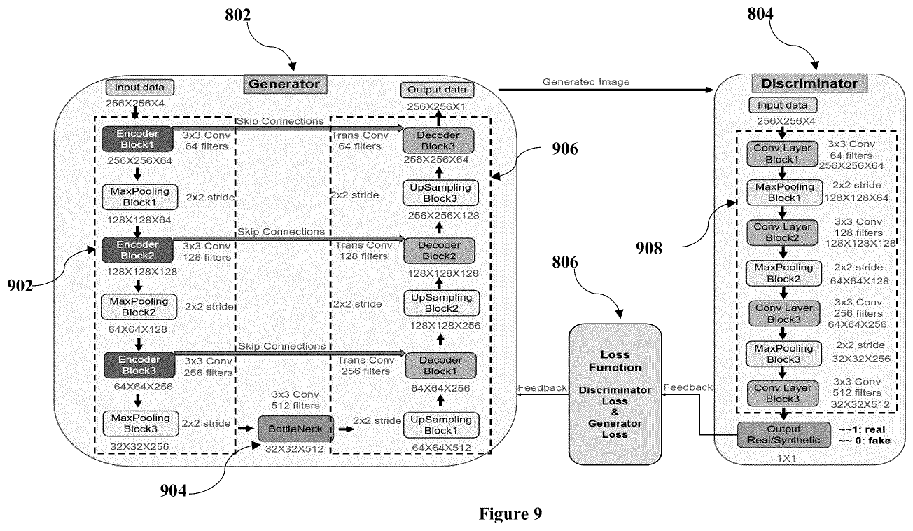

FIG. 9 illustrates a neural network architecture of the second ML model 144, in accordance with an embodiment of the present disclosure. The generator 802 may utilize an encoder-decoder structure, integrating multi-scale skip connections and deformable convolutions to enhance image quality and detail retention. The encoder 902 may include convolutional layers that may extract hierarchical features, reducing spatial resolution while increasing feature dimensionality. This process captures high-level object structures necessary for generating realistic-looking synthetic ultrasound images. The encoder 902 may further include a bottleneck layer 904. The bottleneck layer 904 may compress inputs of the generator 802 into latent representation encoding global contextual information about anatomical features.

The decoder 906 then constructs an image by employing transposed convolutions, while multi-scale skip connections ensure that fine-grained spatial information is preserved. Deformable convolutions within the decoder 906 dynamically adapt receptive fields, improving the capture of intricate details such as soft tissue textures and organ boundaries. The output of the generator 802 may be a high-quality synthetic ultrasound image that maintains anatomical consistency.

The discriminator 804 may be designed using a PatchGAN-based architecture, providing both global and local discrimination capabilities to assess the realism of the synthetic ultrasound images. The discriminator 804 employs convolutional layers 908 to perform patch-level discrimination, ensuring local texture realism, while a global evaluation enforces structural consistency. The discriminator 804 may utilize a loss function 806. The loss function 806 may be output a single scalar value that may represent a likelihood of the synthetic ultrasound image being interpreted as real or synthetic, providing feedback that guides the generator in refining image generation process.

The loss function 806 may output a single scalar value that may represent a likelihood of the synthetic ultrasound image being interpreted as real or synthetic. The output may include discriminator losses, generator losses and total losses while generating the synthetic images 152. The output of the loss function may be provided to the generator 802 as feedback, guiding the generator 802 to produce more authentic and anatomically accurate ultrasound images over successive training iterations.

An essential aspect of this architecture is the multi-scale skip connection between encoder and decoder layers that preserves spatial details, facilitating the retention of structural integrity within the synthetic ultrasound images. These architectural enhancements significantly improve the quality, accuracy, and clinical relevance of the synthetic ultrasound images produced by the second ML model 144.

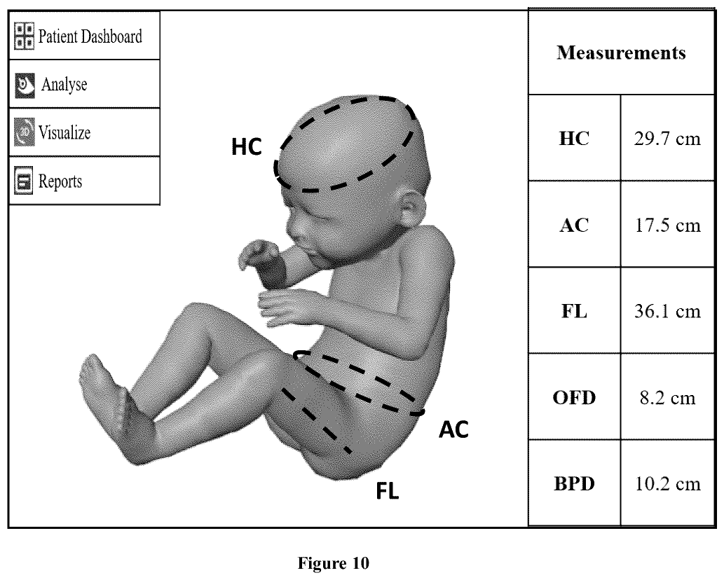

FIG. 10 illustrates an example of an output of the system 100. The output may be provided to the user by a user interface of the user device 102, as shown in FIG. 10. The output includes at least one of the 3D anatomical model and dimensions of the anatomical features present in the ultrasound images provided by the user. In an embodiment, the user interface may provide an option of editing the dimensions, to the user.

TABLE 1

| Models | HC- Dice Score | AC- Dice Score |
|---|---|---|
| MFP U-Net | 97.65 | 94.16 |
| U-Net | 96.97 | 96.14 |
| Dilated U-Net | 97.51 | 95.51 |
| R2U-Net | 92.93 | 84.94 |
| Attention U-Net | 97.43 | 95.43 |

Table 1 illustrates Dice scores of results of different ML models for image segmentation of head (HC) and Abdomen (AC) taken from the MFP Dataset for Fetal Head and Abdomen.

Dice Similarity Coefficient (DSC), also referred to as the Dice score, is a statistical measure used to evaluate similarity between two sets in image segmentation tasks. It quantifies an overlap between a predicted segmentation and a ground truth reference, providing a robust metric for assessing segmentation accuracy. A higher overlap would indicate that the model is working segmenting images accurately. This metric is widely utilized in various applications, including medical image analysis, autonomous systems, and natural language processing, to objectively compare segmentation outputs and improve model performance.

TABLE 2

| Models | Training Data | Dice Score |
|---|---|---|
| First ML model 142 | 174 Samples | 97.45 (Training) |
| | 50 Samples (Head) | 90.05 (Validation) |
| | 50 Samples (Abdomen) | |
| | 74 Samples (Femur) | |

Table 2 illustrates Dice score of result of the first ML model 142 for image segmentation of head, abdomen and femur taken from the MFP Dataset. The dataset included 174 samples—50 samples of head, 50 samples of abdomen and 74 samples of femur. Tables 1 and 2 indicate that the first ML model 142 has a higher Dice score as compared to other ML models.

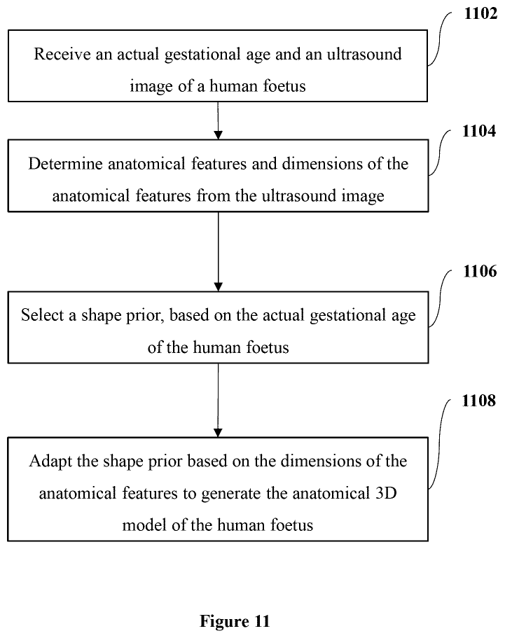

FIG. 11 illustrates a flow chart of a method for generating an anatomical 3D model, in accordance with an embodiment of the present invention. In this regard, each block may represent a module, segment, or portion of code, which comprises one or more executable instructions for implementing the specified logical function(s). It should also be noted that in some alternative implementations, the functions noted in the blocks may occur out of the order noted in the drawings.

For example, two blocks shown in succession in FIG. 11 may in fact be executed substantially concurrently or the blocks may sometimes be executed in the reverse order, depending upon the functionality involved. Any process descriptions or blocks in flow charts should be understood as representing modules, segments, or portions of code which include one or more executable instructions for implementing specific logical functions or steps in the process, and alternate implementations are included within the scope of the example embodiments in which functions may be executed out of order from that shown or discussed, including substantially concurrently or in reverse order, depending on the functionality involved. In addition, the process descriptions or blocks in flow charts should be understood as representing decisions made by a hardware structure such as a state machine.

The order in which method is described is not intended to be construed as a limitation, and any number of the described method blocks can be combined in any order to implement the method. Additionally, individual blocks may be deleted from the methods without departing from the scope of the subject matter described herein.

Furthermore, the method can be implemented in any suitable hardware, software, firmware, or combination thereof. Furthermore, the above-mentioned methods may be implemented in suitable hardware, computer-readable instructions, or a combination thereof. The steps of such methods may be performed by either a system under the instruction of machine-executable instructions stored on a non-transitory computer-readable medium or by dedicated hardware circuits, microcontrollers, or logic circuits. The method may include the following steps.

At step 1102, an actual gestational age and an ultrasound image of a human foetus may be received from a user. The one or more ultrasound images may include, but are not limited to, 2D, 3D, 4D, Doppler, colour Doppler, power Doppler, elastography, transvaginal, transrectal, foetal echocardiography, intraoperative ultrasound, and contrast-enhanced ultrasound (CEUS). The user may include but are not limited to healthcare professionals, researchers, and patients. Further, the user device 102 may include, but are not limited to, desktop computers, laptops, ultrasound machines, and mobile devices.

At step 1104, a first ML model may determine one or more anatomical features and dimensions of the one or more anatomical features from the ultrasound image. The first ML model may be pre-trained using ultrasound images of foetuses. The anatomical features may include, but are not limited to, biparietal diameter, head circumference, abdominal circumference, femur length, occipitofrontal diameter, cerebellar diameter, cisterna magna, nuchal translucency, humerus length, liver and spleen size, placental thickness and position, and tibia and fibula length. The anatomical features are essential for anatomical labelling in the one or more ultrasound images of the foetus.

At step 1106, a shape prior may be selected based on the actual gestational age of the human foetus. The shape prior may be selected from one or more shape priors related to different gestational ages of human foetuses. The one or more shape priors may be pre-constructed using nomogram information.

Shape priors may be reference models that may represent an usual shape and structure of a foetus at different stages of gestation, based on data such as nomograms. The shape priors may help in creation of an accurate 3D model by providing a reference for an expected size and proportions of the foetus at a given age. Further, nomograms may be graphical tools or charts that may represent a relationship between various anatomical features and the gestational age of a foetus. Nomograms may include information on how certain measurements, such as biparietal diameter or femur length, are expected to change as the foetus develops.

At step 1108, the shape prior is adapted based on the dimensions of the one or more anatomical features to generate the anatomical 3D model of the human foetus. The anatomical 3D model may include annotations of the one or more anatomical features and dimensions of the one or more anatomical features.

It must be understood that, although the present invention is described with respect to human foetus, it can be extended to human organs including, but not limited to, heart, liver, kidney, and prostate.

### Technical Advancement and Economic Significance

The method disclosed in the present invention for generating an anatomical 3D model, may have the following advantages over conventional art:
Ensures precise and consistent anatomical assessments of human organs or foetuses by using an ML model.
Overcomes data scarcity by generating synthetic ultrasound images using an ML model.
Generates anatomically accurate fetal and organ models by integrating shape priors derived from nomogram data, ensuring consistency with expected growth patterns.

The specification may refer to “an”, “another”, “one” or “some” embodiment(s) in several locations.

This does not necessarily imply that each such reference is to the same embodiment(s), or that the feature only applies to a single embodiment. Single features of different embodiments may also be combined to provide other embodiments.

The terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps, or acts are in some way inherently mutually exclusive.

As used herein, the singular forms “a”, “an” and “the” are intended to include the plural forms as well, unless expressly stated otherwise. It will be further understood that the terms “includes”, “comprises”, “including” and/or “comprising” when used in this specification, specify the presence of stated features, integers, steps, operations, elements, and/or components, but do not preclude the presence or addition of one or more other features, integers, steps, operations, elements, components, and/or groups thereof. It will be understood that when an element is referred to as being “connected” or “coupled” to another element, it can be directly connected or coupled to the other element or intervening elements may be present. Furthermore, “connected” or “coupled” as used herein may include operatively connected or coupled. As used herein, the term “and/or” includes any and all combinations and arrangements of one or more of the associated listed items.

Unless otherwise defined, all terms (including technical and scientific terms) used herein have the same meaning as commonly understood by one of ordinary skills in the art to which this disclosure pertains. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Although implementations of a method for generating an anatomical 3D model have been described in language specific to structural features and/or methods, it is to be understood that the appended claims are not necessarily limited to the specific features or methods described. Rather, the specific features and methods are disclosed as examples of implementations of a method for generating an anatomical 3D model.

The invention has been described above with reference to numerous embodiments and specific examples. Many variations will suggest themselves to those skilled in this art in light of the above-detailed description. All such obvious variations are within the full intended scope of the appended claims.

We claim:

1. A method (1100) of generating an anatomical three-dimensional (3D) model, comprising steps of:
receiving an actual gestational age and an ultrasound image of a human foetus;
determining, by a first machine learning (ML) model, one or more anatomical features and dimensions of the one or more anatomical features from the ultrasound image, wherein the first ML model is pre-trained using ultrasound images of foetuses;
selecting, based on the actual gestational age of the human foetus, a shape prior from one or more shape priors related to different gestational ages of human foetuses, wherein the one or more shape priors are pre-constructed using nomogram information; and
adapting, by the first ML model, the shape prior based on the dimensions of the one or more anatomical features to generate the anatomical 3D model of the human foetus.

2. The method (1100) as claimed in claim 1, wherein the one or more anatomical features include biparietal diameter, head circumference, abdominal circumference, femur length, occipitofrontal diameter, cerebellar diameter, cisterna magna, nuchal translucency, humerus length, liver and spleen size, placental thickness and position, and tibia and fibula length.

3. The method (1100) as claimed in claim 1, wherein the one or more anatomical features and the dimensions of the one or more anatomical features are determined by:
reducing, by an encoder of the first ML model, spatial resolution of the ultrasound image to determine one or more object features including edges, contours, boundaries, and shapes;
restoring, by a decoder of the first ML model, spatial resolution of the ultrasound image to provide segmented regions of interest;
determining, by the decoder of the first ML model, dimensions of the one or more features in the segmented regions of interest; and
mapping, by the decoder of the first ML model, the one or more object features and the dimensions of the one or more object features with a nomogram to determine the one or more anatomical features and the dimensions of the one or more anatomical features, wherein the nomogram includes information of biparietal diameter, head circumference, abdominal circumference, and femur length of foetuses.

4. The method (1100) as claimed in claim 1, further comprising a step of:
generating, by the first ML model, a report including at least one of:
the ultrasound image comprising labels indicating the one or more anatomical features and the dimensions of the one or more anatomical features; and
an evaluation of foetal growth levels by comparing the anatomical 3D model of the human foetus with nomogram information.

5. The method (1100) as claimed in claim 1, wherein the first ML model is also pre-trained using synthetic ultrasound images generated by a second ML model.

6. The method (1100) as claimed in claim 5, wherein the second ML model includes a pair of generator and discriminator, and
wherein the generator utilizes an encoder-decoder architecture, and the encoder-decoder architecture includes multi-scale skip connections to link layers of an encoder and a decoder to preserve fine-grained spatial details.

7. The method (1100) as claimed in claim 6, wherein the synthetic ultrasound images are generated by the generator using foetal biometry obtained from a nomogram and at least one segmentation mask, wherein the foetal biometry comprises dimensions of anatomical features including biparietal diameter, head circumference, abdominal circumference, femur length, occipitofrontal diameter, cerebellar diameter, cisterna magna, nuchal translucency, humerus length, liver and spleen size, placental thickness and position, and tibia and fibula length, and the at least one segmentation mask includes a spatial layout of the anatomical features.

8. The method (1100) as claimed in claim 7, further comprising steps of:
providing, to the discriminator, the synthetic ultrasound images, and real ultrasound images;
determining, by the discriminator, a loss occurred in generating the synthetic ultrasound images by comparing the synthetic ultrasound images with the real ultrasound images; and
iteratively tuning the generator until the loss falls below a pre-defined threshold value.

9. The method (1100) as claimed in claim 7, wherein the first ML model and the second ML model are selected from a group consisting of a convolutional neural network, recurrent neural network, transformer, generative adversarial network, variational autoencoder, SimCLR, and BERT.

10. A system (100) for generating an anatomical three-dimensional (3D) model, comprising:
a processor (120); and
a memory (140), wherein the memory (140) is communicatively coupled with the processor (120), and the memory (140) stores program instructions executable by the processor (120) to:
receive an actual gestational age and an ultrasound image of a human foetus;
determine, by a first machine learning (ML) model, one or more anatomical features and dimensions of the one or more anatomical features from the ultrasound image, wherein the first ML model is pre-trained using ultrasound images of foetuses;
select, based on the actual gestational age of the human foetus, a shape prior from one or more shape priors related to different gestational ages of human foetuses, wherein the one or more shape priors are pre-constructed using nomogram information; and
adapt, by the first ML model, the shape prior based on the dimensions of the one or more anatomical features to generate the anatomical 3D model of the human foetus.

11. The system (100) as claimed in claim 10, wherein the memory (140) further stores program instructions configured to determine the one or more anatomical features and the dimensions of the one or more anatomical features by:
reducing, by an encoder of the first ML model, spatial resolution of the ultrasound image to determine one or more object features including edges, contours, boundaries, and shapes;
restoring, by a decoder of the first ML model, spatial resolution of the ultrasound image to provide segmented regions of interest;
determining, by the decoder of the first ML model, dimensions of the one or more features in the segmented regions of interest; and
mapping, by the decoder of the first ML model, the one or more object features and the dimensions of the one or more object features with a nomogram to determine the one or more anatomical features and the dimensions of the one or more anatomical features, wherein the nomogram includes information of biparietal diameter, head circumference, abdominal circumference, and femur length of foetuses.

12. The system (100) as claimed in claim 10, wherein the memory (140) further stores program instructions configured to:
generate, by the first ML model, a report including at least one of:
the ultrasound image comprising labels indicating the one or more anatomical features and the dimensions of the one or more anatomical features; and
an evaluation of foetal growth levels by comparing the anatomical 3D model of the human foetus with nomogram information.

13. The system (100) as claimed in claim 10, wherein the first ML model is also pre-trained using synthetic ultrasound images generated by a second ML model.

14. The system (100) as claimed in claim 13, wherein the second ML model includes a pair of generator and discriminator, and
wherein the generator utilizes an encoder-decoder architecture, and the encoder-decoder architecture includes multi-scale skip connections to link layers of an encoder and a decoder to preserve fine-grained spatial details.

15. The system (100) as claimed in claim 14, wherein the synthetic ultrasound images are generated by the generator using foetal biometry obtained from a nomogram and at least one segmentation mask, wherein the foetal biometry comprises dimensions of anatomical features including biparietal diameter, head circumference, abdominal circumference, and femur length, and the at least one segmentation mask includes a spatial layout of the anatomical features.

16. The system (100) as claimed in claim 15, wherein the memory (140) further stores program instructions configured to:
provide, to the discriminator, the synthetic ultrasound images, and real ultrasound images;
determine, by the discriminator, a loss occurred in generating the synthetic ultrasound images by comparing the synthetic ultrasound images with the real ultrasound images; and
iteratively tune the generator until the loss falls below a pre-defined threshold value.

17. A method of generating an anatomical three-dimensional (3D) model, comprising steps of:
receiving an actual age and an ultrasound image of a human organ;
determining, by a first machine learning (ML) model, one or more anatomical features and dimensions of the one or more anatomical features from the ultrasound image, wherein the first ML model is pre-trained using ultrasound images of human organs;
selecting, based on the actual age of the human organ, a shape prior from one or more shape priors related to different ages of human organs, wherein the one or more shape priors are pre-constructed using nomogram information; and
adapting, by the first ML model, the shape prior based on the dimensions of the one or more anatomical features to generate the anatomical 3D model of the human organ.

18. The method as claimed in claim 17, wherein the one or more anatomical features include ventricular ejection fraction, chamber dimensions, and wall thickness of human hearts, liver volume, portal vein diameter, and fat content of human livers, kidney size, cortical thickness, and renal pelvis dilation.

19. The method as claimed in claim 17, wherein the first ML model is also pre-trained using synthetic ultrasound images generated by a second ML model.

20. The method as claimed in claim 19, wherein the synthetic ultrasound images are generated by a generator of the second ML model using organ biometry obtained from a nomogram and at least one segmentation mask, wherein the organ biometry comprises dimensions of anatomical features including ventricular ejection fraction, chamber dimensions, and wall thickness of human hearts, liver volume, portal vein diameter, and fat content of human livers, kidney size, cortical thickness, and renal pelvis dilation, and the at least one segmentation mask includes a spatial layout of the anatomical features.
